(19) United States
(12) Patent Application Publication
(10) Pub. No.: US 2019/0062686 A1
(43) Pub. Date: Feb. 28, 2019

(54) MICROFLUIDIC DEVICE FOR CULTURING CELLS

(71) Applicants: Amin A BANAEIYAN, Göteborg (SE); Mattias GOKSÖR, Mölndal (SE); Caroline B ADIELS, Mölndal (SE); Jannick THEOBALD, Mannheim (DE)

(72) Inventors: Amin A BANAEIYAN, Göteborg (SE); Mattias GOKSÖR, Mölndal (SE); Caroline B ADIELS, Mölndal (SE); Jannick THEOBALD, Mannheim (DE)

(21) Appl. No.: 16/135,015

(22) Filed: Sep. 19, 2018

Related U.S. Application Data
(63) Continuation of application No. PCT/SE2017/050270, filed on Mar. 21, 2017.

(30) Foreign Application Priority Data
Mar. 21, 2016 (SE) 1650371-6

(51) Int. Cl. C12M 3/06; C12M 1/00; C12M 3/00; C12N 5/071; G01N 33/50; B01L 3/00

(52) U.S. Cl. CPC C12M 23/16; C12M 23/34; C12M 21/08; C12N 5/067; B01L 2200/0647; G01N 33/5014; B01L 3/502761; B01L 2200/027; G01N 33/5008

(57) ABSTRACT

A microfluidic device (10) for culturing and/or analysing at least one cell type is disclosed. The device (10) comprises a plurality of chambers (30), arranged in a radial expansion model. Chambers (30) have a central aperture (32) for receiving and/or removing the first cell type into the chamber (30) . The device (10) comprises a wall (40) on the perimeter of each chamber (30), and a feed channel network (50) outside each chamber adjacent to the wall (40) for conveying culture medium, reagents and/or a second cell type. The feed channel network (50) is provided with a central port (51) and configured such that culture medium, reagents, and/or a second cell type provided to the central port are distributed symmetrically in a radial fashion in the feed channel network (50). The wall (40) of the device (10) has a plurality of microfluidic diffusion channels (42) for allowing flow of the culture medium, reagents and/or the second cell type from the feed channel (50) into each chamber (30). Methods of culturing cells using such a device are also disclosed.

This application is the continuation of International Application No. PCT/SE2017/050270, filed 21 Mar. 2017, which claims the benefit of Swedish Patent Application No. 1650371-6, filed 21 Mar. 2016, the entire contents of which are hereby incorporated by reference.

### FIELD OF THE INVENTION

The present disclosure relates to microfluidic devices for culturing and/or analysing cells. More particularly, the disclosure relates to a microfluidic device for culturing and analysing hepatocytes.

### BACKGROUND OF THE INVENTION

Liver-related diseases affect many people worldwide. Each year several thousand new patients join a liver transplant waiting list. Drug-induced liver toxicity is one of the major reasons for drug withdrawals from the market even after long and costly clinical approval procedures are completed. The fact that drug discovery and development heavily relies on animal models leads to high failure rates. The fundamental problem with animal models is that they fail to adequately evaluate and predict mechanisms of liver injury and drug toxicity in humans due to major inter-species genetic variations. More importantly, efficacy and toxicity trials on animal models fail to reveal the specific human metabolic pathways for the substance being tested. The traditional cell culture used in such trials and clinical procedures suffers from several additional drawbacks including being labour intensive and not amenable to process control. This has led to the development of “liver-on-chip” platforms which attempt to better emulate the microphysiological liver environment, in particular the critical liver tissue interfaces and dynamic human physiological complexities.

US 2015/0004077 A1 discloses integrated human organ-on-chip microphysiological systems.

WO 2014/197622 A2 discloses a liver-mimetic device including a 3D polymer scaffold having a matrix of liver-like lobules with hepatic-functioning particles encapsulated within the lobules.

WO 2007/008609 A2 discloses a cell culture unit with a perfusion/medium inlet, a perfusion/medium outlet, a cell loading/reagent inlet, and a waste outlet. The perfusion inlet opposes the perfusion outlet such that medium must pass through each culture unit to proceed to the next, while the cell loading inlet opposes the waste outlet. All inlets and outlets are in the same plane of the unit at approximately right angles to each other. Such a design allows only a small surface area for cells, and may result in less uniform medium being perfused into the culture chamber.

It would be desirable to provide improved microfluidic devices for culturing and/or analysing hepatocytes and other cell types.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a microfluidic device for culturing and/or analysing at least one cell type comprising: a plurality of chambers for a first cell type arranged in a radial expansion model, each chamber having a central aperture for receiving the first cell type into the chamber and/or removing the first cell type from the chamber; a wall on the perimeter of each chamber; and a feed channel forming a feed channel network outside each chamber adjacent to the wall for conveying culture medium, reagents, and/or a second cell type; the feed channel network being provided with a central port and configured such that culture medium, reagents, and/or a second cell type provided to the central port are distributed symmetrically in a radial fashion in the feed channel network; wherein the wall has a plurality of microfluidic diffusion channels for allowing flow of the culture medium, reagents, and/or the second cell type from the feed channel into each chamber. This leads to significantly improved performance than traditional designs where waste/used culture medium must flow through each chamber to progress.

Methods of culturing cells are also provided.

Furthermore, a method of drug efficacy/toxicity testing is provided.

Further advantageous embodiments are disclosed in the appended and dependent patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which

FIGS. 1A and 1B are top views of second layer of the microfluidic device according to an embodiment of the invention.

FIG. 1C is a top view of a microfluidic device according to one aspect of the present invention;

FIG. 1D is a top view of a microfluidic device according to another aspect of the present invention;

FIG. 1E shows perspective views of the microfluidic device of FIG. 1D where the first and layers are separated (left hand side) and connected (right hand side);

FIG. 2A shows an exploded and perspective view of a chamber of the microfluidic device of FIG. 1C;

FIG. 2B shows a top view of the chamber and wall of FIG. 2A;

FIG. 3 illustrates the radial expansion of the chambers of the microfluidic device according to some aspects of the invention;

FIG. 4A-J depicts the fabrication procedure of the microfluidic device of FIG. 1C and the first layer of the microfluidic device of FIG. 1C;

FIG. 5A shows the flow velocity in the feed channel and within the chamber of a microfluidic device according to some aspects of the invention;

FIG. 5B depicts the shear rate in the feed channel and within the chamber of a microfluidic device according to some aspects of the invention;

FIG. 6 illustrates the glucose diffusion pattern within a single chamber and surrounding feed channel;

FIGS. 7A-F shows microscope images of the cell-containing chambers of the microfluidic device according to some aspects of the invention;

FIGS. 8A-C illustrates live- and dead-staining of liver hepatocytes with 4 μM calcein AM and 4 μM ethidium homodimer 5 days after cell seeding of the microfluidic device according to some aspects of the invention.

FIGS. 9A-C further illustrates live- and dead-staining after HepG2 cell seeding of the microfluidic device.

FIG. 10 A, B shows microscope images of the hiPSC-derived hepatocyte cell-containing chambers of the microfluidic device according to an aspect of the invention.

FIG. 10 C shows a 2D culture of cells in a traditional well device.

FIG. 10 D, E shows a 3D culture of cells in a device according to an aspect of the invention.

FIG. 11A is a graph showing the amount of the liver-specific biomarker, albumin, secretion [ng/h/1 M cells] within a period of 5 days for both pump-driven and gravity-driven cultures. n=4 for pump-driven experiments and n=2 for gravity-driven experiments.

FIG. 11B is a graph showing the synthesis of urea [ng/h/1 M cells] in the hepatocyte culture as a functionality measure for hepatocytes during a period of 5 days in culture. n=4 for pump-driven experiments and n=2 for gravity-driven experiments.

FIG. 12A is a graph showing albumin secretion [ng/h/1 M cells] of a culture comprising HepG2 cells over a period of 14 days.

FIG. 12B is a graph showing urea synthesis [ng/h/1 M cells] in a culture of cells comprising HepG2 cells over a period of 14 days.

FIG. 12C is a graph showing albumin secretion [ng/h/1 M cells] of a culture of cells comprising hiPSC hepatocytes over a period of 21 days. Albumin secretion was compared between static 2d cultures and a cell culture provided in the microfluidic device according to an aspect of the invention.

FIG. 12D is a graph showing urea synthesis [ng/h/1 M cells] in a culture of cells comprising hiPSC hepatocytes over a period of 21 days. Urea synthesis was compared between static 2 d cultures and a cell culture provided in the microfluidic device according to an aspect of the invention.

FIG. 13 is a top view of a wall of a microfluidic device according to an aspect of the invention wherein part of the walls are formed by a plurality of posts.

FIG. 14A shows formation of bile-canaliculi in a culture of cells in a device according to an aspect of the invention.

FIG. 14B shows insignificant formation of bile-canaliculi in a monolayer culture of cells.

FIG. 15A shows the metabolism of testosterone in cultures provided on a 96-well plate in comparison to cultures provided in a device according to an aspect of the invention.

FIG. 15B shows the metabolism of testosterone in cultures co-treated with

Rifampicin provided on a 96-well plate in comparison to cultures provided in a device according to an aspect of the invention.

FIG. 15C shows the metabolism of Diclofenac in cultures provided on a 96-well plate in comparison to cultures provided in a device according to an aspect of the invention.

FIG. 16A-E shows albumin secretion in cultures prepared in wells and a device according to an aspect of the invention, both non-treated controls and drug treated are shown.

FIG. 17A-E shows urea synthesis in cultures prepared in wells and a device according to an aspect of the invention, both non-treated controls and drug treated are shown.

### DETAILED DESCRIPTION

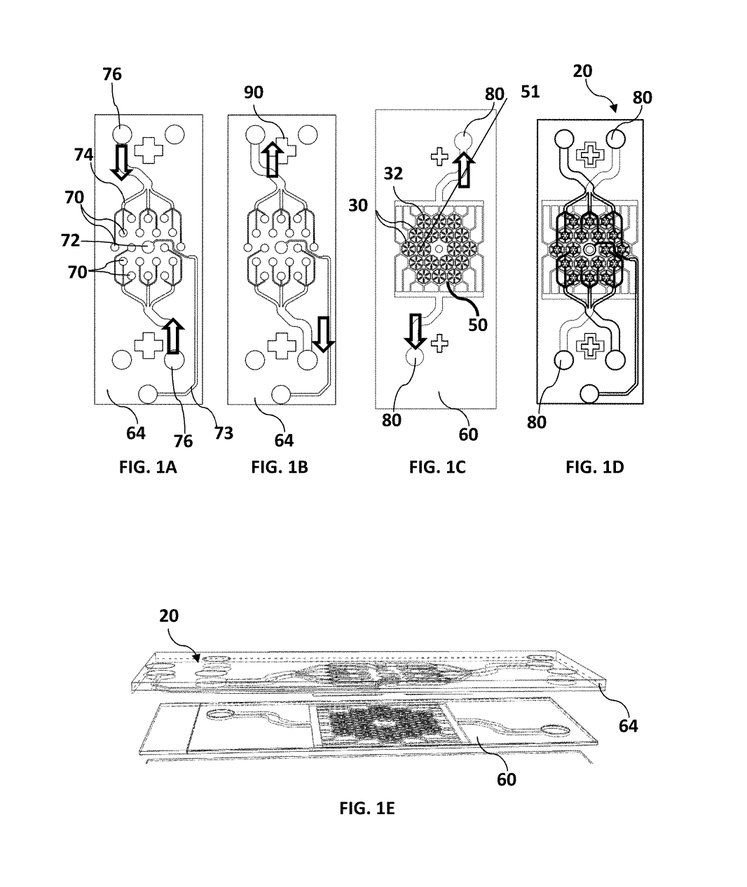

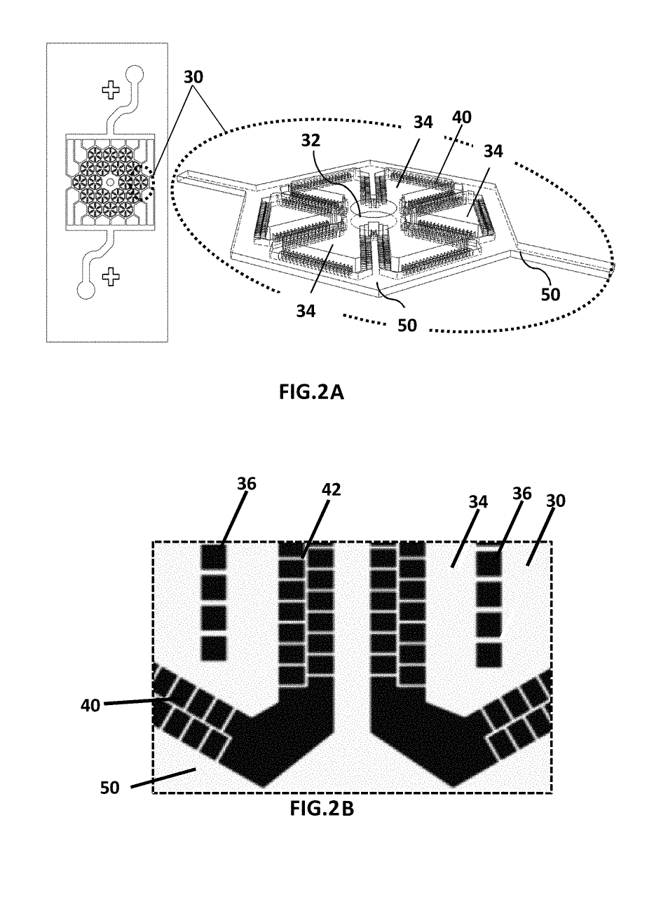

The following description focuses on embodiments of the present invention applicable to a microfluidic device for culturing and analysing hepatocytes. However, it will be appreciated that the invention is not limited to this application but may be applied to many other cell types for example, kidney cells, heart cells, pancreatic cells, endothelial cells, Kupffer cells, liver endothelial cells, stellate cells. The device is also suitable for co-culturing cells of different types. The device may be a liver-lobule mimetic. FIGS. 1 and 2 illustrate microfluidic devices 10 and 20. The microfluidic devices 10, 20 each comprise a plurality of chambers 30 for culturing the hepatocytes. Each chamber 30 is substantially sealed at a lower portion. Each chamber 30 has a central aperture 32 for receiving the hepatocytes into the chamber 30 and/or removing the hepatocytes from the chamber 30 as will be described further below. The central aperture is arranged at the radial centre of the chamber 30, that is, not at the perimeter of the chamber 30. The central aperture 32 may be provided in an upper portion of the chamber 30. The upper region of the chamber 30 may be at least partially sealed. The upper region of the chamber 30 may be sealed except for the central aperture 32 such that the chamber 30 is substantially sealed. A wall 40 is present on the perimeter of each chamber 30 for separating the hepatocytes in chamber 30 from a feed channel 50 located outside each chamber 30 and adjacent to the wall 40. In some instances, the wall 40 is not provided with apertures for the provision of cells. It has been shown that the provision of cells via a central aperture 32 located in the upper region of the chamber 30 and not at a side wall leads to improved cell culturing. The feed channel 50 has a network-type layout around the plurality of chambers 30 for conveying culture medium and/or reagents. The feed channel 50 forms a feed channel network 50 adjacent to and in connection with the walls 40 of a plurality of chambers 30. Outlets 80 are located towards the periphery of the devices 10, 20 for receiving the culture medium and/or reagents after they have passed through the feed channel 50. The feed channel network 50 comprises a central port 51 for receiving culture medium, reagents, or as is disclosed below, additional cells to be cultured. The central port 51 is provided at the radial centre of the feed network 50. Each chamber 30 may be formed by an arrangement of cell-culture compartments 34 arranged in a flower-petal like arrangement. The flower-petal arrangement may comprise 6 cell-culture compartments 34. The flower-petal arrangement is formed by a plurality of, such as six, compartments 34 formed as lobules extending from a central region of the chamber 30 radially outward. The chamber 30 design results in 3D tissue formation inside the culture chambers. As is disclosed below, the central aperture 32 enables a very high density of cells to be delivered to, and cultivated within, the chambers. On receipt of cells via the central aperture 32 the chamber volume is substantially filled with cells. Each chamber 30 can have a height of between 40 μm and 90 μm, such as approximately 60 μm. This enables at least 3 layers of cells to be cultured within each chamber 30. Due to close cell-to-cell contact and interaction a 3D tissue-like structure is achieved in contrast to a monolayer of cells. This feature promotes the cell integrity and in vivo-like functionality of the cells. This is not observed in a traditional 2D monolayer of cell cultures. Cell supernatant, cell secretion and any drug metabolites may be collected from the central aperture 32 of the chambers 30. In this way the central aperture 32 mimics the central vein of the liver-lobule.

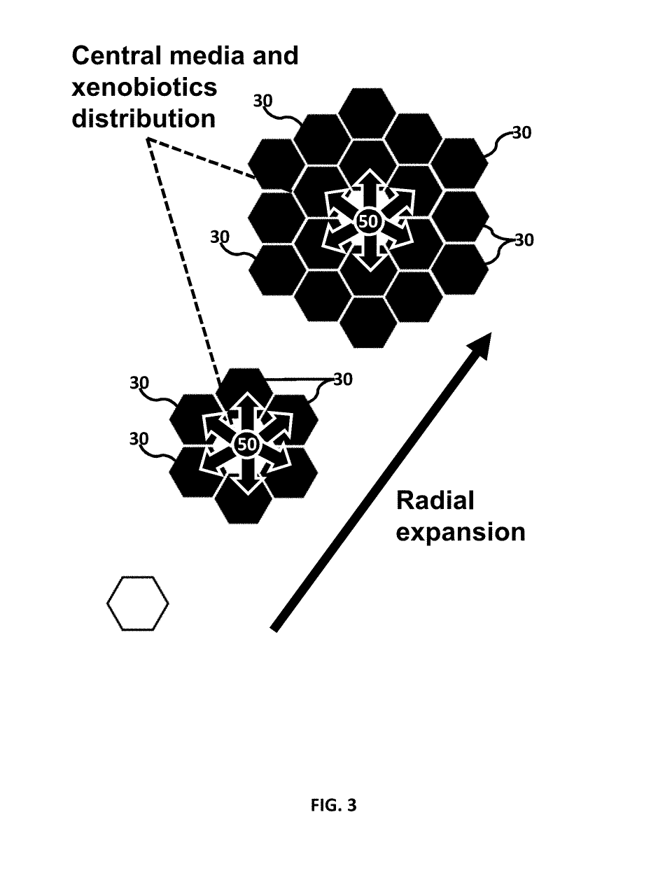

The chambers 30 are arranged in a radial expansion model as shown in FIGS. 1, 2 and 3. The chambers 30 are arranged in concentric rings. Each chamber 30 is adjacent to a plurality of other chambers 30. As shown in FIG. 3, a chamber having 6 sides is adjacent to from two to six other chambers 30. The number of chambers 30 adjacent to a respective chamber 30 is dependent on in which concentric ring it is located.

The feed channel network 50 extends between adjacent chambers 30. As with the chambers 30, the feed network 50 extends in a radial expansion model. The feed channel network 50 extends radially outward from the central port 51 to the periphery of the plurality of chambers 30. The feed channel network 50 may form a honeycomb pattern channel surrounding the chambers 30. A fluid provided to the central port 51 of the feed channel network 50 is distributed symmetrically in a radial fashion extending from the central port 51 to the radially outermost parts (i.e., the parts being adjacent to the chambers 30 which form the outermost concentric ring) of the feed channel network 50. The radial flow allows distribution of the fluid to the radially outermost parts of the feed channel network 50, adjacent to the outermost chambers 30, regardless of the number of chambers 30 present in the device 10, 20. The fluid channel network 50 is sealed at its upper portion. The feed channel network 50 is sealed at its lower portion. The feed channel network 50 is continuous. That is, fluids distributed in the feed channel network do not need to pass through a first chamber 30 to reach a subsequent chamber 30. In other words, the flow within the feed channel network 50 is substantially convective. An advantage of the feed channel network 50 is that it allows better control of the provision of culture medium and therein better performance. The feed channel network 50 also means that all culture chambers receive fresh medium. This leads to significantly improved performance than traditional designs where waste/used culture medium must flow through each chamber to progress.

As can be seen in FIG. 2, the wall 40 has a plurality of microfluidic diffusion channels 42 for allowing flow of the culture medium and/or reagents from the feed channel network 50 into each chamber 30. The microfluidic diffusion channels 42 have a width of from 1 μm to 20 μm, such as from 2 μm to 10 μm, preferably from 2 μm to 7 μm, or about 2 μm and a depth of from 1 μm to 20 μm, such as from 2 μm to 10 μm, preferably from 2 μm to 7 μm, or about 2 μm to protect the cells from high sheer rate of the convective flow through feed channel network 50. The wall 40, and in particular the diffusion channels 42, are dimensioned such that cells are substantially held within the chamber and cannot pass through the wall 40. Without wishing to be bound by theory, it is believed microfluidic diffusion channels 42 represent fenestrated endothelial cells of the liver lying alongside the entire lobule sides. The microfluidic diffusion channels 42 allow for the diffusion of the nutrients and xenobiotics to the hepatic tissue while protecting hepatocytes from the convective shear flow. The wall may also be a dual-wall structure as shown in FIG. 2. The dual-wall structure comprises a first longitudinal wall part and second longitudinal wall part arranged adjacent to one another. Both longitudinal walls parts have a plurality of diffusion channels 42 where the diffusion channels are offset from one another, that is, the channels 42 are not aligned axially.

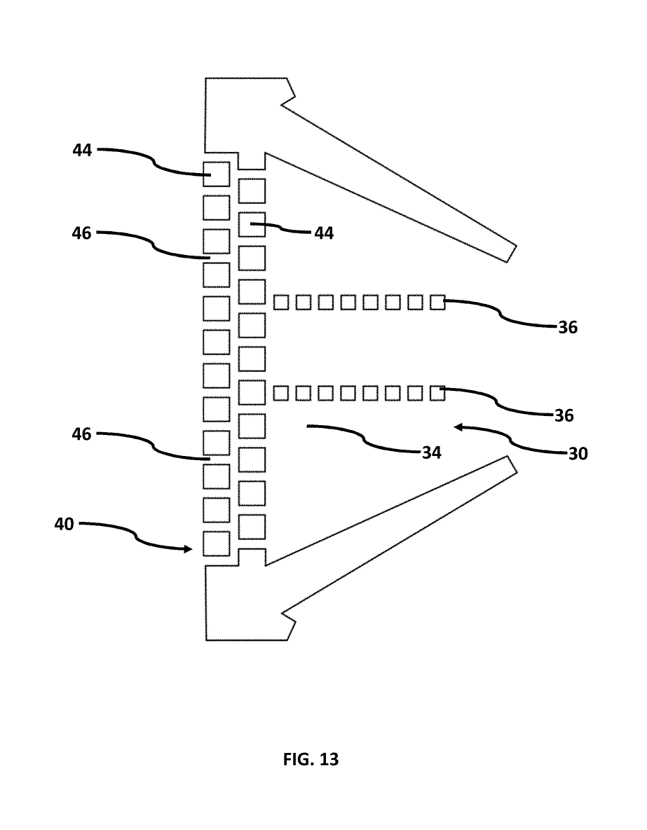

FIG. 13 shows a schematic of a portion of a wall 40 adjacent to one compartment 34 of a chamber 30. As shown in FIG. 13 a portion(s) of the walls 40 may also comprise a plurality of posts 44 arranged such that diffusion channels 46 are formed between the posts 44 through which culture medium and/or reagents from the feed channel 50 may diffuse into chambers 30. Like the microfluidic diffusion channels 42, the diffusion channels 46 limit the shear rate in the chamber 30. The diffusion channels 46 are less than 10 μm wide, such as about 5 μm wide. As is evident from FIG. 13 however, diffusion channels 46 are wider than microfluidic diffusion channels 42 as shown in FIG. 2. Although the diffusion channels 46 are wider than the microfluidic diffusion channels 42, the walls 40 in the device of FIG. 13 are still able to hold the cells in the chamber area 30. An advantage of such an arrangement is that the diffusion channels 46 allow limited contact between cells provided on either side of the wall 40. Such direct cell-to-cell contact may enhance the suitability of the device for co-culturing of different cell types. This will be described further below for example, in respect of hepatic cells in the chamber 30 and fibroblasts in the feed channel network 50.

The wall 40 concentrates the hepatocytes in the chamber 30 and minimizes the convective flow through the chambers 30 while allowing diffusive transport. Each chamber 30 comprises a plurality of cell culture compartments 34 extending radially from the central aperture 32 towards the wall 40. The devices 10, 20 comprise a plurality of free-standing posts 36 in each chamber 30, preferably in the cell culture compartments 34. Posts 36 provide a large surface area support for the hepatocytes in the chambers 30. Posts 36 also prevent the chamber wall sagging and provide a mechanical grip for the freshly seeded cells to attach and align the tissue-like structures in a radial orientation. The posts may be substantially cubic such that a larger surface area is provided for cell adhesion. The wall 40 and the posts 36 comprise a biocompatible polymer. For example, they can comprise, such as consist of polydimethylsiloxane (PDMS), but other suitable polymeric materials such as polymethylmethacrylate (PMMA), polycarbonate (PC), or polystyrene (PS) may be used. The dimensions of each chamber are generally intended to be bio-relevant or biomimetic. For example, the diameter of the chamber may be similar to the diameter of to the diameter of a mammalian liver lobule, such as a human liver lobule. The dimensions may be from about 1 mm to about 2.5 mm, such as about 1.2 mm to about 2.4 mm. Each chamber has a cell culture array with a large surface area for cell adhesion.

Microfluidic device 10 is formed on a single layer 60 while microfluidic device 20 has two layers i.e. first 60 and second 64 layers. In device 20 the plurality of chambers 30, wall 40, and feed channel network 50 are located on the first layer 60. The second layer 64 is located above the first layer 60 as shown in FIG. 1E. The second layer 64 comprises a plurality of openings 70 coinciding with each central aperture 32 for feeding the hepatocytes into the chamber 30 and/or receiving the hepatocytes and supernatant of the cells from the chamber 30. An inlet 72 is located centrally on the second layer 64 and coincides with the feed channel 50 to provide the culture medium, nutrients, reagents, or xenobiotics to the feed channel network 50. The inlet 72 coincides with the central port 51 of the feed channel network 50. The nutrient flow is then distributed symmetrically in a radial fashion in the bottom feed network 50 towards outlets 80 in accordance with the arrows in FIG. 1C. The hydraulic resistance of the fluidic network formed by feed channel 50 is balanced to ensure the equal flow rates on all sides of the chambers 30. Each opening 70 has a channel 74 extending away from the opening 70. Some of the channels 74 merge into one larger channel that leads to a main opening 76 on the periphery of the second layer 64. Two main openings 76 are shown in FIG. 1A. Opening 76 may be connected to a hepatocyte source or waste container (not shown). The main opening(s) 76 are initially used for hepatocyte cell seeding in accordance with the arrows in FIG. 1A. After the hepatocyte loading step is finished the channels 74 are washed with fresh medium and then openings 70 and channels 74 drain the chambers 30 as shown by the arrows in FIG. 1B. The inlet 72 may also be a combined inlet/outlet port 72. The port 72 can receive supernatant, cell secretion and/or drug metabolites (FIG. 1B). This inlet or inlet/outlet port the functionality of the central vein of a liver lobule. The inlet/outlet port 72 may be in cooperation with, such as connected to, a channel 73.

The microfluidic devices 10, 20 allow for precise control over fluid flow to create an in vivo circulation mimetic, a very large surface area of the tissue that can be expanded radially, a separate feeding network on the top layer (when present) to create different feeding layouts independent of the bottom tissue culture layer, radial flow distribution of culture medium in the feed channel network, multiple tissue culture chambers that can be reached through an integrated top feed network on a single chip, cost effective replica production of the devices 10, 20, system compatibility with both pump-driven and gravity driven flow profiles, and possibility of integration in multi-organ platforms.

FIG. 3 shows how the microfluidic device may be constructed with differing numbers of chambers 30 by a radial expansion model. The dimensions may be adjusted as necessary. Thus, in some embodiments the microfluidic device comprises at least 6 chambers, such as between 6 and 100 chambers. In some embodiments a system of at least two microfluidic devices 20 such as those described herein may be provided wherein each of the devices 20 comprises a plurality of chambers 30. The system of devices may be arranged such that a first device may be used for culturing one cell type. A second device may be connected either serially or in parallel with the first device. The cell-types cultivated in each device 20 may be the same, or may be different. The ease of manufacturability of the devices allows for several devices 10, 20 to be manufactured together in a single process. In such a system the devices 10, 20 may be connected such that different concentrations of a drug may be provided to each separate device 20. The different concentrations of drugs may be provided via a gradient flow system arranged in cooperation with channel 73 and inlet/outlet port 72. In such a system, the channel 72 may connect at least two of the devices 20. The channel 72 may connect the at least two devices 20. Drug toxicity or efficacy experiments comprising a range of different concentrations can therefore simply be performed with a single system comprising more than one device 20.

As stated above the device is suitable for culturing a variety of cell types and not exclusively hepatic cells. The device may also be used for culturing brain cells (neurons, glial cells), cardiac muscle cells (cardiomyocytes), lung epithelial cells (alveolar), intestinal epithelial cells, ovarian cells, fat cells (adipocytes), renal proximal tubule epithelial cells, bone marrow cells, liver endothelial cells, capillary blood vessel cells, brain endothelial cells, lung endothelial cells, fibroblast cells, retinal vascular endothelial cells, kidney (renal) cell, Kupffer cells, hepatic stellate cells or microvascular endothelial cells. The device may be used for culturing cancer cell lines such as mammary cancer cells or liver cancer cells (HepG2, HepaRG). Parenchymal cells which in vivo are subject to limited shear stress and low flow rates may be cultured in the chamber 30. Stromal cells, or macrophages, which in vivo are subject to higher shear stress and higher flow rates may be cultured in the feed channel 50. The device is also suitable for co-culturing cells of two or more different types. For example, hepatic cells may be cultured in the chamber 30 whilst fibroblasts may be cultured in the feed channel 50. The first cell type may be cultured in a low shear flow environment, that is a region of the device where the velocity of flow is decreased and the shear flow thus also decreased, preferably the low shear flow environment is the chamber 30. The low shear flow in the chamber 30 is shown in the flow simulation section below. The second cell type may be cultured in a higher shear flow environment. The term higher shear flow environment is intended to mean a higher shear flow relative to the low shear flow present in regions of the device, such as the chamber 30. The higher shear flow environment is preferably the feed channel 50.

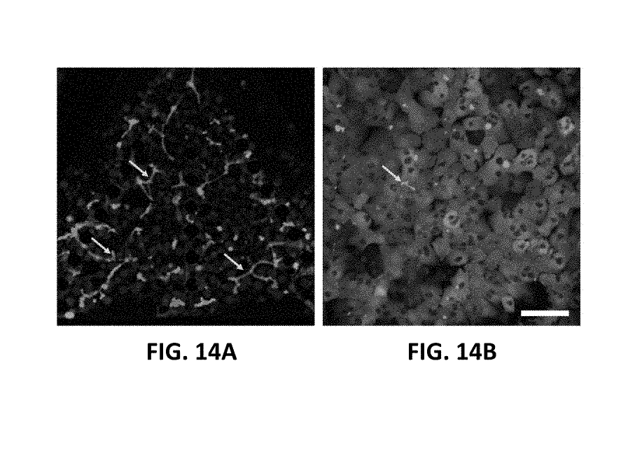

When used for co-culturing cells the cells present in low shear flow environment, e.g. the chamber 30, may be in direct cell-to-cell contact or may be hindered from having direct cell-to-cell contact with the cells in the higher shear flow environment, e.g. feed channel 50. For example, in the device shown in FIG. 13 direct cell-to-cell contact is possible as the diffusion channels 46 are large enough that cells present in the chamber 30 can contact cells present in the feed channel 50. However, in the device shown in FIG. 1 having micro diffusion channels 42, this direct cell-to-cell contact is not possible as the micro diffusion channels 42 are not large enough to permit any cell present in the chamber 30 to contact any cell in the feed channel 50. A second cell type, such as NIH-3T3, endothelial cells may be introduced into feed channel 50 via the inlet 72. As endothelial cells require shear stress for improved functionality this is an ideal culture environment while the media passes on top of the endothelial cells whereas hepatocytes are protected within chamber 30 from the direct convective flow in feed channel 50. A porous polymer (eg., PE or PDMS) layer may be introduced between the layers of the device. This allows for culturing a third or fourth etc. cell type stacked on top of each other in the feed channel 50. Such 3D cell cultures as described herein, whether of one or several heterogenous cell types, may be considered a microtissue. FIG. 14A shows the formation of bile canaliculi in a 3D culture of cells in a device according to the invention. The bile canaliculi are marked with an arrow. In a monolayer culture, insignificant formation of bile canaliculi occurred as can be seen in FIG. 14B. The formation of such structures supports the 3D and in-vivo like nature of the cell culture in devices according to the invention.

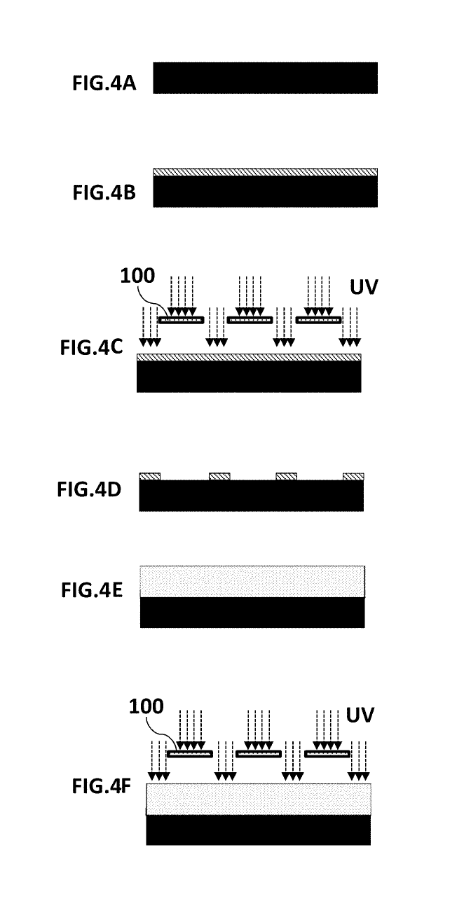

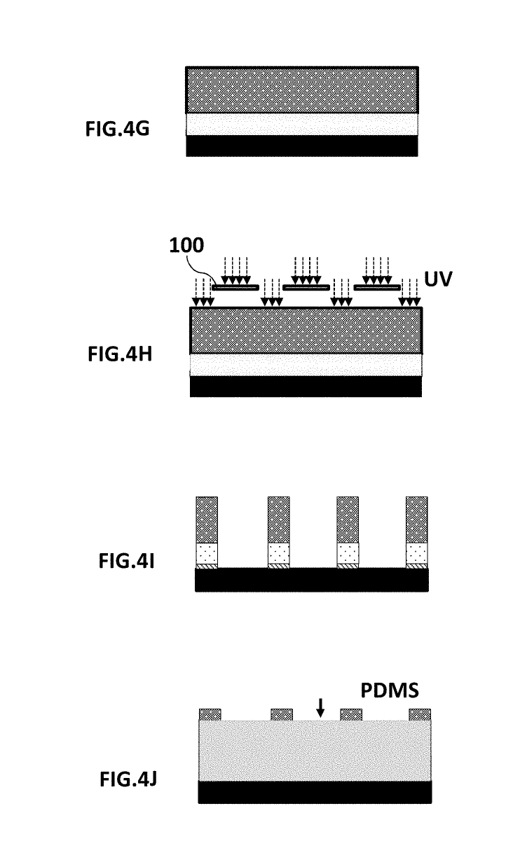

The fabrication process of the bottom tissue culture layer 60 is shown in FIG. 4. To fabricate the plurality of chambers making up bottom tissue culture master a 3-layer coating approach was used. Initially a 3-step Acetone-Isopropyl Alcohol (IPA)-Methanol cleaning on the 4-inch silicon wafers was performed (FIG. 4A). The wafers were sonicated for 5 minutes in acetone before transferring into IPA. After the wafers were dried with pressured nitrogen a 15-minute dehydration step at 200° C. was performed. Wafers were cooled to room temperature. The first thin layer was spin-coated for the diffusion channels using SU8-2002 photoresist (microchem). The photoresist was coated at 500 rpm for 5 seconds and then at 3000 rpm for 30 seconds according to the manufacturer's protocol (FIG. 4B). These coating settings yielded a layer with a cross-section of 2 μm×2 The wafer was soft-baked at 65° C. for 1 minute and then at 95° C. for 5 minutes. The wafer was cooled to room temperature and then exposed in a mask aligner for 3 seconds at 6 mW/cm² using the diffusion layer mask set. After post exposure bake (PEB) at 65° C. for 1 minute and at 95° C. for 3 minutes the wafer was developed in mr-Dev600 for 30 seconds (FIG. 4C). The wafer was washed multiple times with de-ionized water (DIW) and dried with a nitrogen gun.

To fabricate the feed channel network on the layer the processed wafer was coated with SU8-2035 at 500 rpm for 10 seconds and then at 1000 rpm for 30 seconds (FIG. 4D). A single coating with these settings provided a 60 μm-thick photoresist layer as measured by dektak surface profiler. After the coating step the wafer was soft baked at 65° C. and 95° C. for 5 and 25 minutes respectively. The coated wafer was exposed in a mask aligner for 15 seconds. The channel layer mask was aligned to the diffusion channel thin layer using the alignment marks 90 on the wafer. The alignment marks 90 are shown in FIGS. 1B and 1C. The exposed wafer was post exposure baked at 65° C. for 5 minutes and at 95° C. for 30 minutes. This layer was not developed. Instead the wafer was coated again with SU8-2035 at 500 rpm for 10 seconds and then 600 rpm for 30 seconds successively for 4 times with a soft-bake step in between each coating. The wafer was soft-baked at 65° C. for 5 minutes and at 95° C. for 30 minutes. These coating settings provided a thick 400-μm stencil layer. The stencil layer mask was then aligned to the two previous layers and the wafer was exposed for 60 seconds to UV radiation (FIG. 4E). Afterwards a PEB step at 65° C. and 96° C. for 10 minutes and 1 hour respectively was performed. Finally the 5 stacked SU8-2035 layers were developed for 45 minutes with occasional agitations (FIG. 4F). The wafer was rinsed with DIW multiple times and then hard baked at 160° C. for 20 minutes. The final hard bake step reflowed the minor cracks in the thick photoresist layer and added to the chemical and mechanical stability of the final structures. The purpose of the thick stencil layer was to fabricate cylindrical pillars that coincide with the central aperture 32 of each chamber 30. This will be described further below.

The fabrication of the top feeding and seeding layer 64 followed the same procedure as explained above for the tissue culture layer 60. One layer of SU8-2035 was spin-coated at 500 rpm for 10 seconds and then 600 rpm for 30 seconds. This provided a layer with an approximate thickness of 100 μm. The wafer was soft-baked at 65° C. for 5 minutes and 95° C. for 30 minutes. The wafer was exposed with the top layer mask for 15 seconds. A PEB at 65° C. for 5 minutes and at 95° C. for 30 minutes was performed. The wafer was developed for 15 minutes in the developer, rinsed with DIW and dried with a nitrogen gun. A hard bake at 160° C. for 20 minutes was completed. To fabricate a single microfluidic device in PDMS, PDMS mixture was prepared separately for the first and second layers (bottom and top layers respectively). The PDMS:crosslinker ratio was 5:1 for the bottom layer and 15:1 for the top layer. These two ratios ensured proper adhesion between the two layers to prevent leakage during the long-term experiments. The PDMS mixture was spin-coated on the bottom layer silicon master at 200 rpm for 45 seconds to fabricate the culture chambers 30, walls 40 and bottom feed network channels 50. The lower level of PDMS compared to the 400 μm-thick stencil pillars made it possible to readily generate the central aperture 32 for each chamber 30. This way a precise central aperture 32 can be made for each chamber 30 without the need for manual punching. This also allows shrinking down of the size of the chambers 30 as no puncher needle was used. The spin-coated wafer was degassed for 30 minutes and observed for air bubbles. The wafer was baked for minimum 2 hours at 90° C. in a conventional oven. Microfluidic devices were carefully peeled off from the wafer and cut into the desired size. The outlet holes were punctuated by a 3-mm puncher.

To fabricate the top layer the 15:1 PDMS mixture was poured over the top feeding and seeding master layer, the wafer was degassed for 30 minutes and then baked at 90 ° C. for at least 3 hours. The PDMS layer was detached from the wafer after cooling to room temperature and cut into the same sizes as the bottom layer. The openings 70, 76 were punched with a 2 mm puncher.

Microscope glass slides (22×76 mm, 1 mm-thick) were vigorously washed in IPA and then rinsed in IPA and 70% ethanol to clean the surface from organic residues and remove debris. Washed glass slides were placed in the plasma-bonding chamber. Consecutively the bottom thin membrane was washed the same as the glass slides, dried with a nitrogen gun and placed in the plasma chamber. The surfaces of the glass and PDMS were treated with air plasma at 18 W RF power for 30 seconds. The two surfaces were then permanently bonded together. The bonded device was placed in a 90° C. oven for 1 hour to enhance the bonding by thermal treatment. After the device was brought to room temperature the surface of the PDMS was cleaned with Scotch® tape to remove PDMS residues and debris and the device was placed in the bonding chamber again. The top PDMS layer was washed with the previously mentioned procedure, dried with a nitrogen gun, and placed in the plasma chamber. After plasma treatment with the same method the two layers were carefully aligned on top of each other using the designated alignment marks 90. The complete microfluidic device was placed in a 90° C. oven for the final 1-hour bake.

Experiments

Experiment 1—Cell Seeding/Culturing and Maintenance of the Microfluidic Devices

Both ipsc and primary hepatocytes were cryopreserved and directly thawed prior to seeding.

Seeding/Culturing of Cellartis hiPSC

Enhanced ips derived hepatocytes were purchased from Cellartis (Takarabio, Gothenburg, Sweden) and were handled according to the company's protocol. Briefly, cells were thawed in a 37° C. water bath and immediately transferred to 15 ml of thawing medium (InvitroGro HT from BioreklamationIVT)+0.1% PEST and Y-23627. Each vial contained approximately 12 M viable cells. Cells were incubated in the thawing medium at room temperature for 15-20 minutes and centrifuged at 100×g for 2 minutes. The thawing medium was aspirated and cells were gently re-suspended in plating medium (InvitroGro CP Bioreklamation IVT)+0.1% PEST. 96 well plates were immediately seeded by 150 pi of the cell suspension and placed in the incubator. To seed the devices, cell suspension was centrifuged again at 100×g for 2 minutes. The entire plating medium was aspirated and the cell suspension was adjusted to the desired concentration of 5×10⁶ cell s/ml.

Primary cells from two different donors were used. The cells were obtained from BioreklamationIVT and were stored in liquid nitrogen. The vials were thawed in 37° C. water bath immediately prior to the seeding step. Cells were incubated for 15 minutes in 15 ml of thawing medium (InvitroGro CP from BioreklamationIVT)+1% PEST. After centrifuging at 100×g for 2 minutes the thawing medium was aspirated and cells were transferred to the maintenance medium (InvitroGro HI BioreklamationIVT)+1% PEST. 96 wells were seeded immediately with 70 pi of the cell suspension and the rest of the tube was centrifuged at 100×g for 2 minutes to obtain the 5×10⁶ cells/ml cell concentration. The seeding procedure was the same as ips-derived cells.

Seeding/Culturing of iCell hiPSC-Derived Hepatocytes

Cryopreserved hiPSC-derived hepatocytes (iCell Hepatocytes 2.0) were purchased from CDI. iCell Hepatocytes 2.0 were plated according to the manufacturer's protocol. 75 ml of plating medium RPMI 1640 (1X)+GLUTAMAX™ (Gibco, Thermo Fisher Scientific) was supplemented with 1.5 ml B27 supplement 50X (Thermo Fisher Scientific), 20 ng/ml Oncostatin M (R&D Systems, Minneapolis, USA), 0.1 μM Dexamethasone (Thermo Fisher Scientific), 25 μgml-1 Gentamicin (Thermo Fisher Scientific) and 1.5 ml iCell Hepatocytes 2.0 medium supplement (CDI). The cell vial was thawed at 37° C. in a water bath for 3 min and contents immediately transferred into 10 ml of 37° C. plating medium. After centrifuging the cell suspension at 200 g for 3 min, cell pellet was resuspended in fresh plating medium and adjusted for the desired seed concentrations. The devices and 96 wells were pre-coated with collagen I (Sigma-Aldrich, Germany) according to the manufacturer's protocol. Afterwards, cell-seeding devices and wells were placed in the incubator for 4 h. Fresh plating medium was replaced for the wells and a flow at 1 μl/min started for the devices afterwards. Medium change was performed on a daily basis until day 5. After day 5, plating medium was replaced by maintenance medium. The maintenance medium formulation contained all the supplements in the plating medium except Oncostatin M.

HepG2 cells

HepG2 cells were obtained in cryopreserved vials (Sigma Aldrich Gmbh) and were cultured in mammalian cell facilities. Cells were cultured at 37° C. with 5% CO₂. Cell vials were thawed in 37° C. water bath for 2 minutes and immediately transferred to pre-warmed RPMI 1640 (1×)+ GLUTAMAX™ cell culture medium (Gibco, Thermo Fisher Scientific)+1% PEST (Hyclone, Thermo Scientific)+10% FBS (Hyclone Thermo Scientific). Cells were cultured in 75 cm² culture flasks (Sarstedt, Germany) for 4 days to 80% confluency. The media in the flask was changed every other day. On the day of seeding, cells were washed with PBS (-Ca, -Mg) (GE Healthcare HyClone) and detached from the culture flask by adding 1 ml of trypsin/EDTA (GE healthcare). Cells were transferred to 5 ml of fresh medium and centrifuged at 200×g for 3 minutes. Supernatant was aspirated and the cell pellet was re-suspended in fresh medium and adjusted to the concentration of 5×10⁶ cells/ml.

A negative pressure of 3 psi was applied to the media inlet to create a mild low pressure in the channels and to remove air from the tissue chambers. For short-term cultures no ECM coating was used. In the case of long-term experiments cells were mixed in a 1:1 ratio with 20% Geltrex® (Thermo Fisher Scientific) solution and infused into the seeding inlets. After cell loading, seeding channels were flushed with culture medium and devices were inspected under a light microscope (Olympus, CKX41, Japan). To enhance the cell adhesion, the devices were filled with medium and placed inside the cell incubator for 12 h in no-flow conditions.

Experiment 2—Cell Morphology and Long-Term Maintenance, Comparison Between Ipsc, Primary Hepatocytes and HepG2 Cell Line

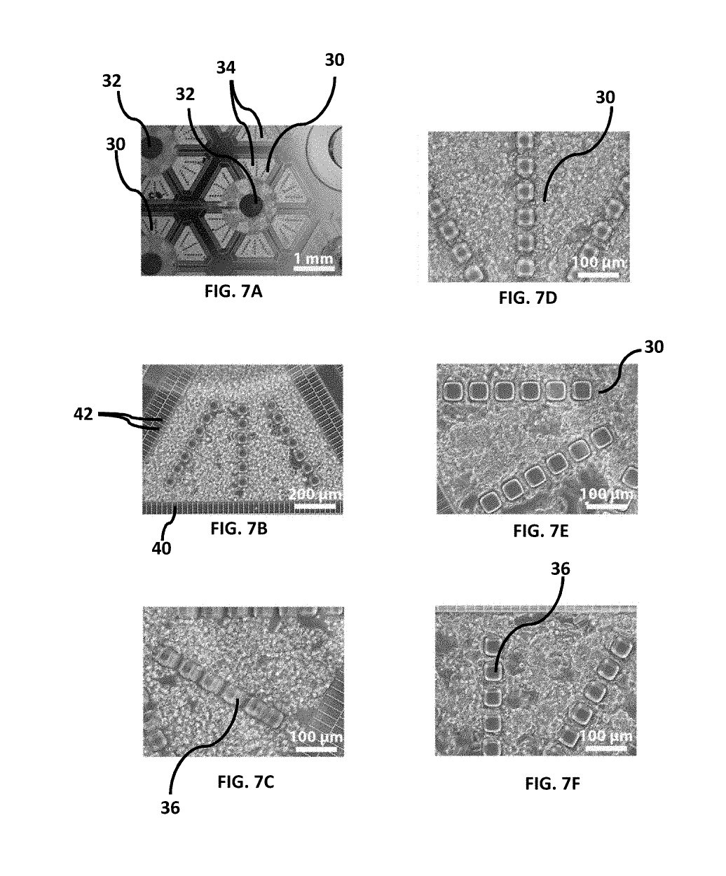

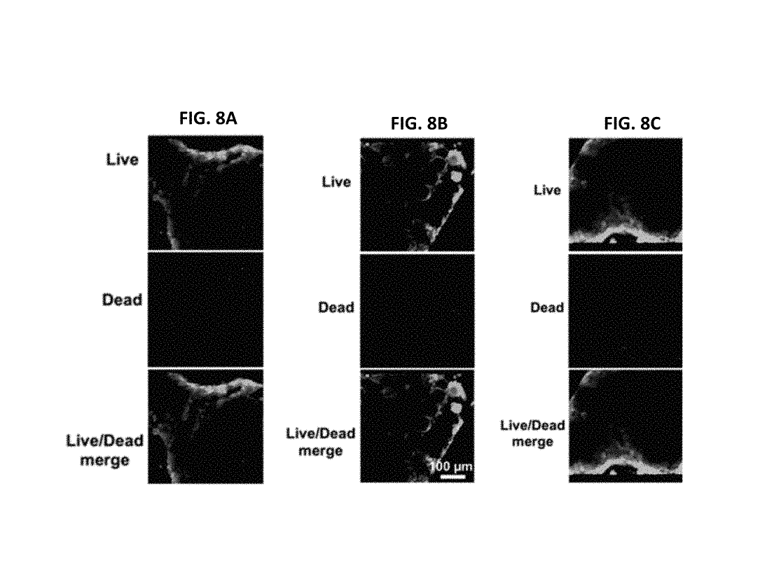

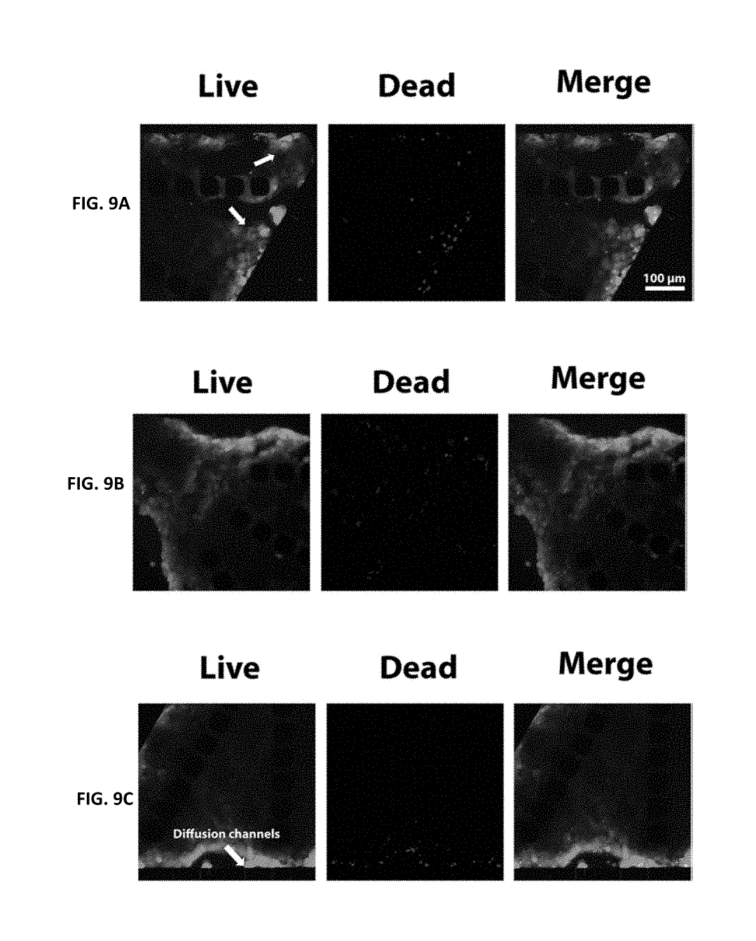

FIG. 7 shows the tissue morphology of HepG2 cell line in the tissue chambers 30. Images are taken in day 5 after cell seeding. The cluster formation and tissue-like structure generation was observed starting from day 2 after seeding. The duration of experiments was 6 days for HepG2 cells. For ips-derived hepatocytes it was observed that during the 3 weeks after cell seeding day the cells form the 3D tissue-like structures (Data not shown). The tissue formation process started at day 2 after seeding after cells were attached to the bottom glass slide. This process was monitored on a daily basis and bright-filed microscope images were taken every second day. Primary cells did not attach to the bottom of the glass slide without an extra cellular matrix (ECM) coating and remained as cell clusters during the 7 days of experiment period (Data not shown). To monitor the cell viability in the culture chambers a Live/Dead assay kit from life technologies was used. Calcein AM in two concentrations and ethidium homodimer were used at the final concentration of 4,6 μM and 4 μM respectively to stain the cells. An epi-fluorescent microscope stage (DMI 6000B, Leica Microsystems, Wetzlar, Germany) was used to probe the fluorescent emission signal from the cells. The concentration of the dyes was optimized to get the strongest signal from the cells while minimizing the background florescence. To stain the cells, the microchips were washed with PBS (-Ca, -Mg) for 5 minutes in a flow rate of 1 μm/min. 4 μm calcein AM and 4 μM ethidium homodimer were dissolved and mixed in PBS. Cells were stained under a 15-minute flow rate interval for a total time of 45 and 90 minutes, while being incubated at 37° C. Microchips were then washed with PBS at 1 μl/min for an extra 5 minutes. Chips were evaluated under the microscope and both brightfield and fluorescent images were taken from several lobules. FIG. 8 shows the viability of HepG2 cells 5 days post seeding in culture. A green fluorescent signal (Calcein AM) shows the live cells and a red signal (Ethidium homodimer) shows the dead cells. For reproduction, the red and green colours may be omitted from FIGS. 8-10. In FIG. 8 the images of the live cells are labelled “Live” in the top row and the images of the dead cells are labelled “Dead” in the middle row. The bottom row shows an image composite of the “Live” images on the “Dead” images. In FIGS. 9 the live cells are labelled “live” in the first column, whilst the dead cells are labelled “dead” in the middle column, the third column shows a composite of the “live” and “dead” images.

To qualitatively assess the formation of bile canaliculi in hepatocyte cultures CDFDA (5-and-6)-carboxy-2′,7′-dichlorofluorescein diacetate (Sigma-Aldrich, Germany) was used at a final concentration of 10 μM. The dye was diluted in PBS and infused into the feeding channel or directly added to the 96-well cultures. Cells were incubated for 10 min at 37° C. and observed for bile formation. Exertion of bile is the hepatocyte-specific role in the liver.

HepG2 Cells

After cell seeding in both short-term (5 days) and long-term (14 days) experiments with HepG2 cells, microfluidic devices were placed in the incubator for cell attachment for 12 h under static conditions. Brightfield images were taken to monitor cell morphology after seeding, as seen in FIG. 7(a). All culture chambers were observed for cell density and air bubbles. FIG. 7(a)-top shows multiple lobules immediately after cell seeding. Optimized cell density was imperative to maximize the cell-to-cell interactions as seen in FIG. 7(a)-middle. Individual cells could be identified after the seeding step (FIG. 7(a)-bottom). Formation of tissue-like structures was observed on day 1 post seeding. Morphology of the tissue in different lobules on day 3 is shown in FIG. 7(b).

The same morphology and 3D tissue formation was observed in the long-term cultures (data not shown).

The viability of HepG2 cells was evaluated on day 4 as seen in FIG. 8. Results showed that cells remained viable throughout the experiments. Long-term cultures were stained with higher 6 μM calcein AM on days 4, 7, and 14 and incubated for 90 min.

Results indicated that penetration of calcein AM to the core of cell clusters was improved significantly. HepG2 cells were stained with CDFDA for bile formation on different days of the culture; however, no distinct bile-canaliculi network was detected in our experiments (data not shown).

hiPSC-Derived iCell Hepatocytes 2.0

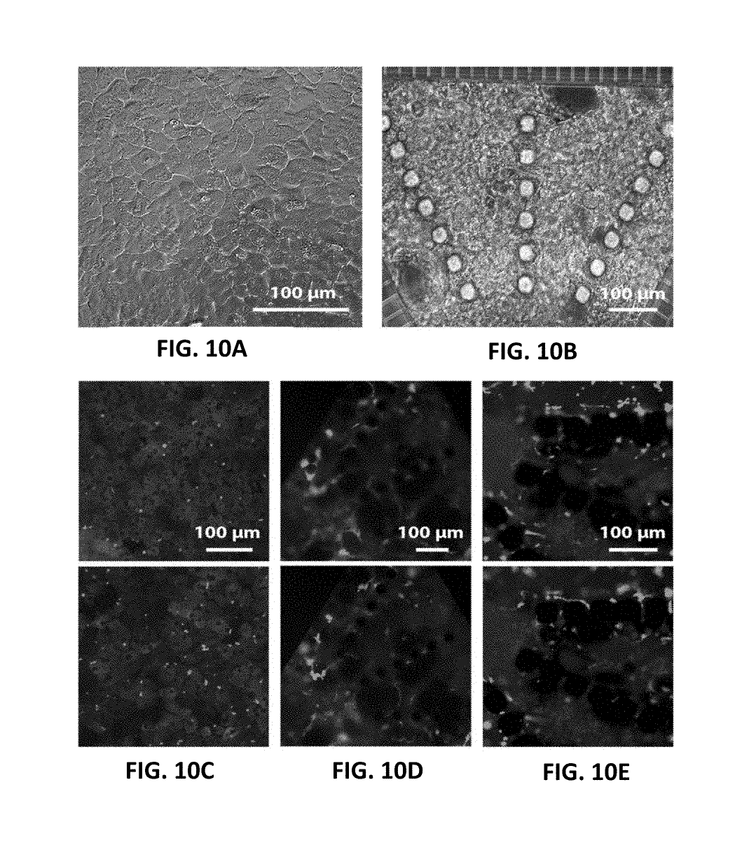

Cell morphology for iCell Hepatocytes 2.0 was monitored during the culture period using a brightfield microscope on a daily basis. In 2D culture plates cells displayed an adherent monolayer and cobblestone like morphology within 48-72 h post plating and retained this morphology throughout the experiments (FIG. 10(a)). In the devices the formation of 3D clusters was observed within 48 h post seeding and the structures became more distinct after day 7 (FIG. 10(b)). hiPSC-derived cells were examined for functional bile-canaliculi formation by using fluorescent dye CDFDA. A functional bile-canaliculi network was observed on day 8 in cells cultured in microfluidic chips, and confirmed on day 16 (FIGS. 10(d), (e)) indicating the preservation of the differentiated cell phenotype, whereas these structures were absent in 2D monolayer cultures (FIG. 10(c)). FIG. 14A shows hiPSC-hepatocytes stained with CDFDA. The arrows indicate the exerted bile to the canaliculi network. FIG. 14B shows a monolayer culture with insignificant bile canaliculi formation.

Assays

Levels of albumin secretion as the main liver biomarker and urea as a major measure of hepatocyte functionality were quantified and normalized per hour to the sample volume and seeded cells.

Experiment 3—Albumin Secretion Assay

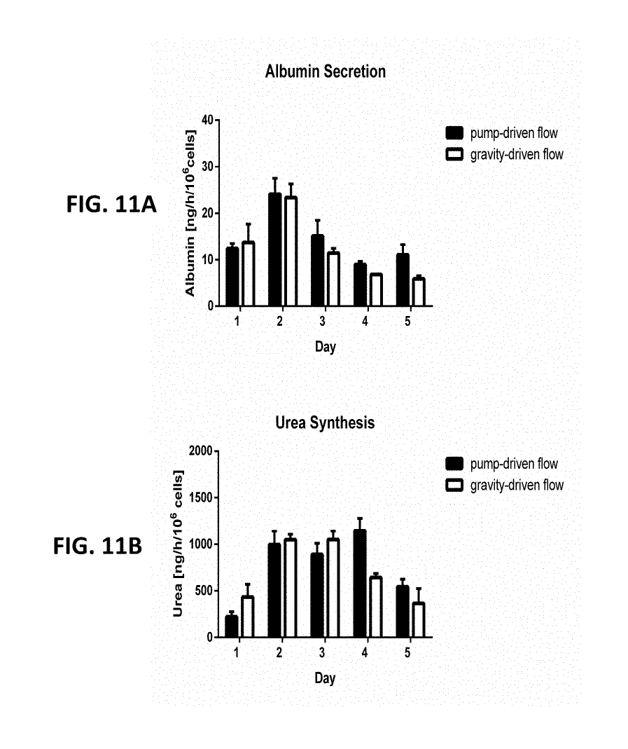

Albumin secretion as a liver-specific biomarker was measured by means of enzyme-linked immunosorbent assay (ELISA) from Bethyl Inc. The assay was performed based on the manufacturer protocol. Collected supernatants were stored in −20° C. prior to the assay day. ELISA assays were run in clear flat bottom 96 well plates (Nunc™) and measured in a microtiter plate reader (FLUOstar Omega, BMG LABTECH, Germany) in absorbance mode at 450 nm wavelength. As seen in FIG. 11(a) the amount of secreted albumin per day for a period of 6 days was recorded for HepG2 cells under both pump-driven and gravity-driven conditions. The results show that the amount of secreted albumin for the devices under a steady flow condition was higher compared to the gravity-driven flow devices. However, by elaborating the top feed network and adjusting the hydraulic resistance of the feed channels a steady gravity-driven flow with desired flow rates under the whole 24-hr period may be achieved.

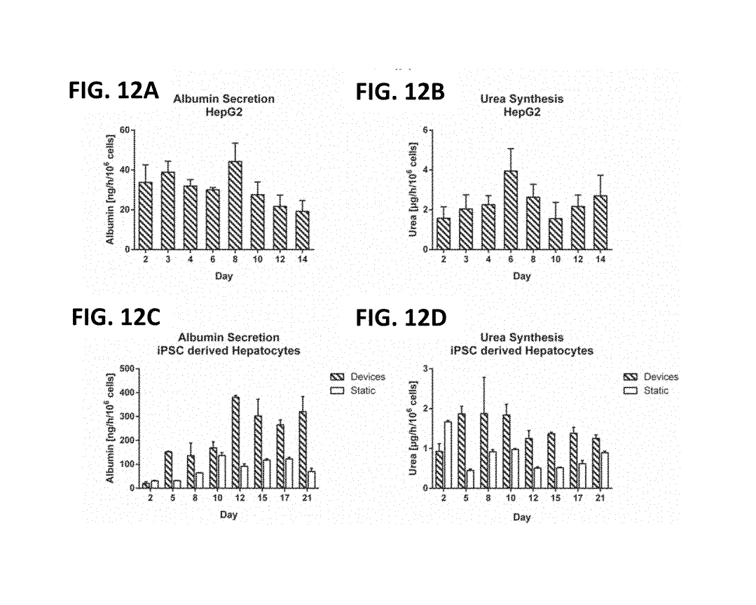

For long-term cultures of HepG2 and hiPSC-derived hepatocytes albumin secretion and urea synthesis were observed for 14 and 21 days, respectively. As seen in FIG. 12(a), HepG2 cells maintained the levels of albumin for 14 days with the maximum metabolic activity during the first 10 days in the culture. For iCell Hepatocytes 2.0 albumin secretion was increased with the progression in cell maturation and differentiation starting on day 5. As seen in FIG. 12(c), peak albumin was detected in the second week of the culture around 400 ng/h/106 cell and levels remained stable until day 21. A 2-5-fold higher albumin secretion in the devices compared to static 2D well cultures was observed.

Experiment 4—Urea Synthesis Assay

Urea synthesis from the cells was used as a measure of cell functionality. Supernatants were prepared according to the manual from the urea assay kit obtained from sigma Aldrich (Sigma Aldrich, MAK006). Urea assays were run in clear, flat bottom 96 well plates (Nunc™) and measured in the microtiter plate reader in absorbance mode at 570 nm wavelength.

For HepG2 cells, the experiments showed as depicted in FIG. 11B, that steady or increasing levels of urea were maintained during the culture period (assayed for 5 days after cell seeding). Urea synthesis was used as a measure for hepatocyte functionality. Over the longer term, urea synthesis (FIG. 12(b)) increased slightly at the end of the first week of culture and maintained a level around 2 μg/h/10⁶ cells.

For iCell Hepatocytes urea synthesis remained between 1.3-1.8 μg/h/10⁶ cells throughout the culture period in the devices and was significantly higher compared to 2D well cultures (FIG. 12(d)).

Experiment 5—Drug Metabolism

Metabolism of Testosterone

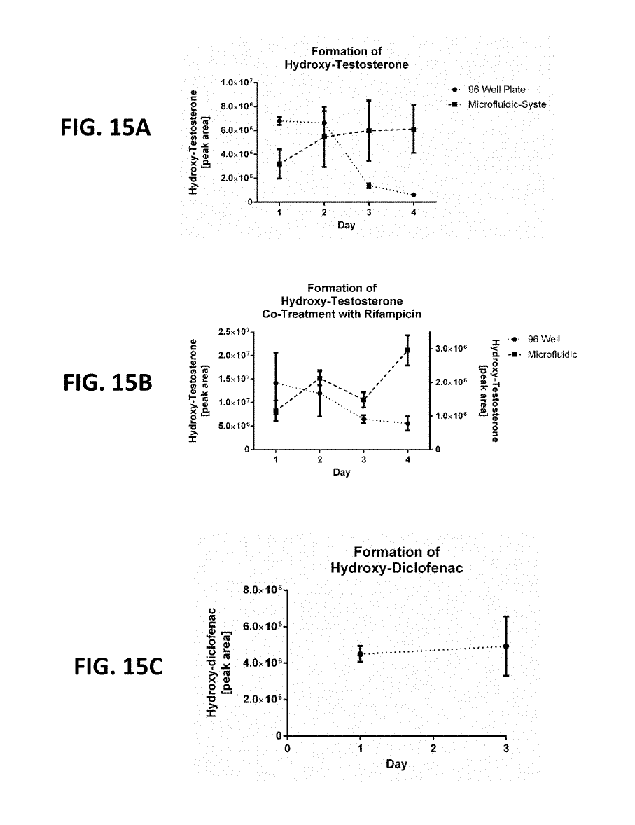

Cells were cultured as described above in a device according to the invention, and in a traditional 96-Well plate. On days 10 to 14 the cells were dosed with testosterone and the formation of hydroxy-testosterone, the primary metabolite of testosterone was measured. Results are shown in FIG. 15a (days 1-4 of dosing shown in graph).

Metabolism of Testosterone and Rifampicin Co-Administration Cells were cultured as described above in a device according to the invention, and in a traditional 96-Well plate. On days 10 to 14 the cells were dosed with testosterone and Rifampicin and the formation of hydroxy-testosterone, the primary metabolite of testosterone was measured. Rifampicin (inducer) and testosterone (substrate) are metabolized by CYP 3A4 enzyme group. Results are shown in FIG. 15b (days 1-4 of dosing shown in graph).

Metabolism of Diclofenac Sodium Cells were cultured as described above in a device according to the invention, and in a traditional 96-Well plate. On days 10 to 14 the cells were dosed with Diclofenac sodium of hydroxy-Diclofenac, the primary metabolite of Diclofenac sodium was measured. Diclofenac sodium us metabolized by CYP 2C9 enzyme group. Results are shown in FIG. 15c (days 1-4 of dosing shown in graph).

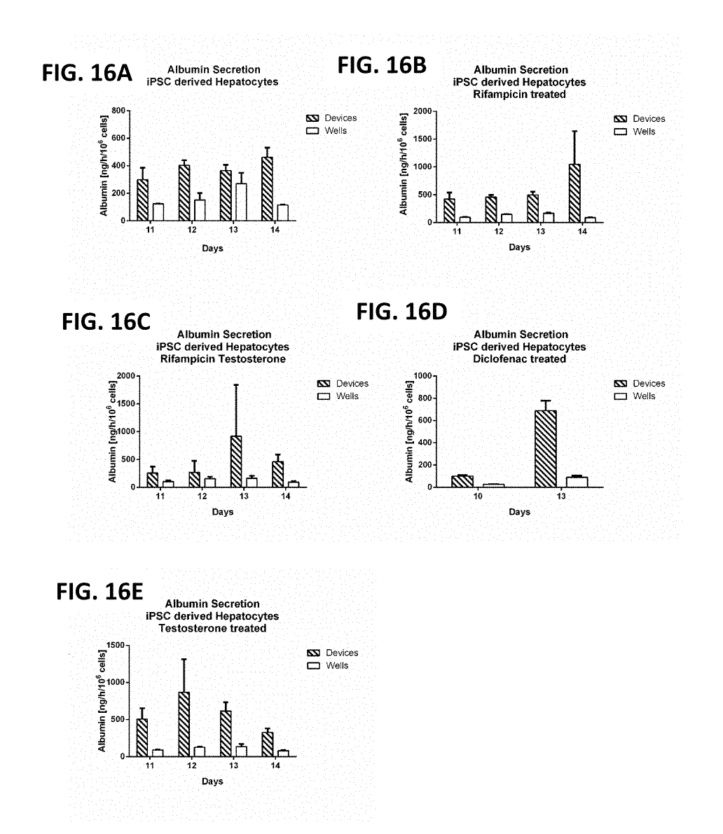

FIGS. 16A-E show albumin secretion in cell cultures, both non-treated controls and drug treated cultures, compared between devices according to the invention and traditional well plates.

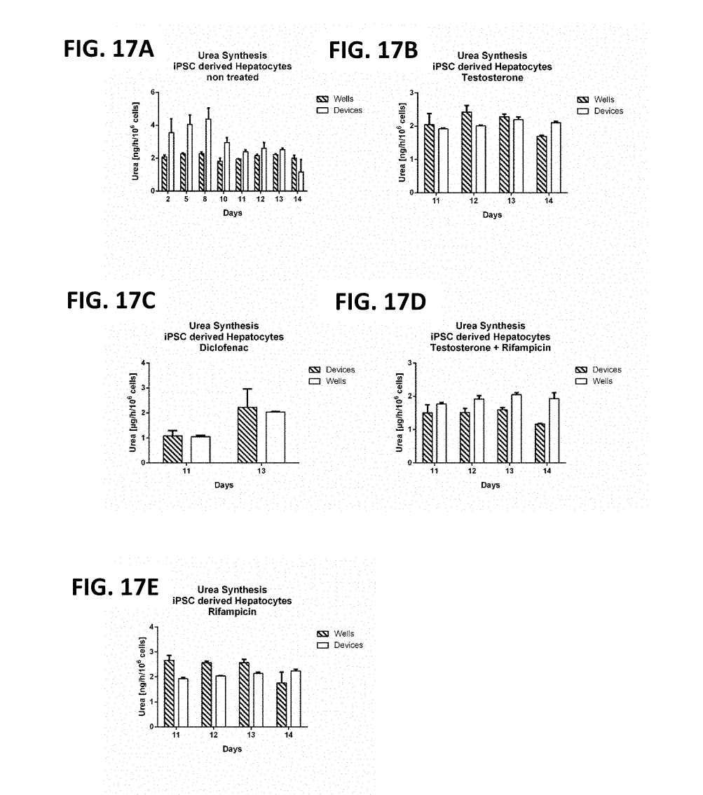

FIGS. 17A-E show urea synthesis in cell cultures, both non-treated controls and drug treated cultures, compared between devices according to the invention and traditional well plates.

Experiment 6—Different Flow Conditions

Experiments were conducted under several different flow conditions to determine the effect of the flow profile and the residence time of the flow in the channels 50 on hepatocyte functionality. Without wishing to be bound by theory, it was hypothesized that a constant controlled flow of the fresh cell media will deliver nutrition and oxygen to the cells while removing the secreted cell waist through the central aperture 32 an ultimately to the main openings 76. In turn, this should promote cell survival and maintenance of the cells in a better condition.

The following flow conditions to the devices were employed:
Gravity-driven: Flow condition during the volume displacement between inlet and outlet reservoir.
Pumping: Constant flow condition with intervals of 15 minutes flow, 15 minutes steady in a total 24-hour time period

In the gravity-driven arrangement, feeding was performed through a reservoir in the inlet 72 of the device, which provided the possibility of a flow for as long as required such that that media exchange between the inlet and outlet reservoirs balanced the height of the fluid on both sides. In the constant pumping syringe flow, the pump was set up for intervals of 15 minutes flow and 15 minutes no flow. All devices were kept in 37° C. and 5% CO₂ condition. The supernatant collection was performed every 24 hours.

Simulation 1—Flow Simulations and Diffusion in the Chambers 30 and Feed Channel 50

COMSOL multiphysics finite element simulation software (COMSOL inc.) was used to simulate the fluid flow in the microchannels. The device geometry for a single module of the lobule tissue chambers 30 was imported into COMSOL environment. The “fine” physics controlled mesh was selected for the finite element simulations. Newtonian, incompressible flow was selected under the no-slip boundary conditions at the channel walls. All channel cross sections were rectangular. To model the flow velocity and shear rate in the bottom hexagonal-shaped feed channel network 50 and inside the tissue chambers 30 the single-phase “Laminar Flow” module was used in the software and introduced a continuous flow rate of 1 μl/min to the device inlet. The pressure of the device outlet was set to be 0 Pa. The fluidic behaviour of the flow in the laminar regime was simulated under the assumption of the constant fluid density and mass conservation and governed by the Navier-Stokes equation (I):

$$ρ ( \frac{δ u}{δ t} + u \cdot ∇ u ) = - ∇ p + η {∇}^{2} u + f \qquad ( 1 )$$

with u being the flow velocity, ρ=0.9933 gr/cm³ and η=0.692×10⁻³ Pa·s the density and the dynamic viscosity of the fluid at 37° C. respectively. P is the pressure and f denotes the other body forces assumed to be “zero” in the simulations. The diffusion of glucose, as the main ingredient present in the culture medium, through the diffusion channels was also simulated. The concentration of glucose was set to 1 gr/litre or 5.5 mol/m³. From Buchwald, P., A local glucose-and oxygen concentration-based insulin secretion model for pancreatic islets. (Theon Biol Med Model 2011, 8, 20) the diffusion coefficient for glucose at 37° C. was set to be D=9e⁻¹⁰ m²/s. The diffusion was assumed to be governed by the standard stationary convection-diffusion equation

$$( \frac{\partial c}{\partial t} = 0 ) \text{:}$$

∇.(−D∇c)=R−∇.(uc) (2)

where c is the concentration of the species [mol.m⁻³], D is the diffusion coefficient [m².s⁻¹], R is the reaction rate [mol.m⁻³.s⁻¹], u is the velocity [m.s⁻¹], and ∇ the del operator. For diffusion studies the “Transport of Diluted Species” module was used and the simulations run under the time-dependent conditions.

The Flow, Sheer Stress and Diffusion Simulations

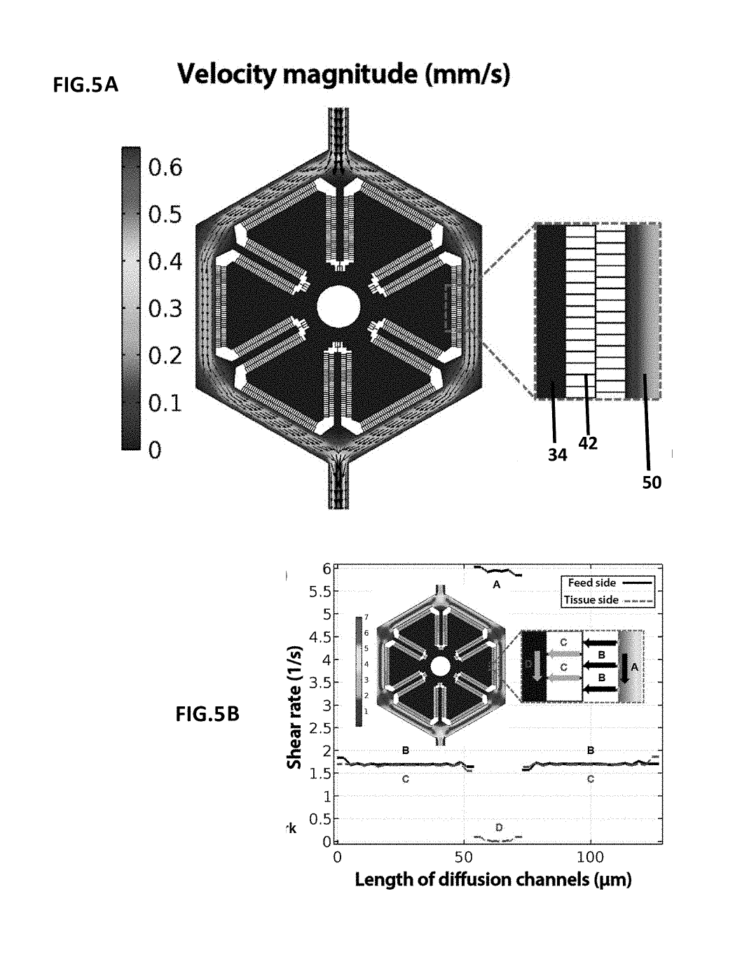

Using the flow simulation conditions mentioned above the flow velocity was simulated in the bottom feed network 50 and inside the tissue chambers 30 as shown in FIG. 5A. The velocity magnitude was found to be 0.3 mm/s and 8e⁻⁴ mm/s in the feed and diffusion channels respectively. Based on these values the Reynolds number was calculated using equation (3).

$$Re = \frac{ρ {uD}_{H}}{η} \qquad ( 3 )$$

In (3), DH is the hydraulic diameter for a channel with rectangular cross section and is calculated to be around 92 μm for the feed channels and 2 μm for the diffusion channels using equation (4). The Reynolds number was calculated in the bottom feed channel network 50 to be around 0.04 at the flow rate of 1 μl/min. The Re number in the microfluidic diffusion channels 42 was found to be 23e⁻⁷. Using equation (5) the Peclet number as a measure of convective/diffusive flow was calculated to be around 30 for the main feed network and 1.8e⁻³ for the diffusion channels indicating a dominant diffusive mass transport through the tissue chambers.

$$\begin{matrix}{D}_{H} = \frac{4 ( w + h )}{2 wh} & ( 4 ) \\ Pe = \frac{{uD}_{H}}{D} & ( 5 )\end{matrix}$$

In equation (4) w is the width and h is the height of the rectangular channel. In equation (5) D is the diffusion coefficient.

FIG. 5B shows the shear rate of the flow in the feed channel network 50, inside the chambers 30, and through the microfluidic diffusion channels 42. The shear rate of the flow was found to be around 6 s⁻¹ in the feed side while being ≈0 on the tissue side (inside the chamber 30). The shear rate alongside the diffusion channels 42 was around 1.7 s⁻¹. This translates to an equivalent shear stress of around 0.04 dyne/cm², 0.01 dyne/cm² and 5×10⁻⁰⁴ dyne/cm² for the feed channels 50, alongside the diffusion channels 42 and the interior side of the tissue chamber 30 respectively as calculated by equation (6):

τ=γη (6)

with τ being the shear stress and γ the shear rate. The wall 40 and the microfluidic diffusion channels 42 of the microfluidic device protect the cells from the high shear force of the flow and facilitate adhesion and long-term functionality of the hepatocytes or other cells.

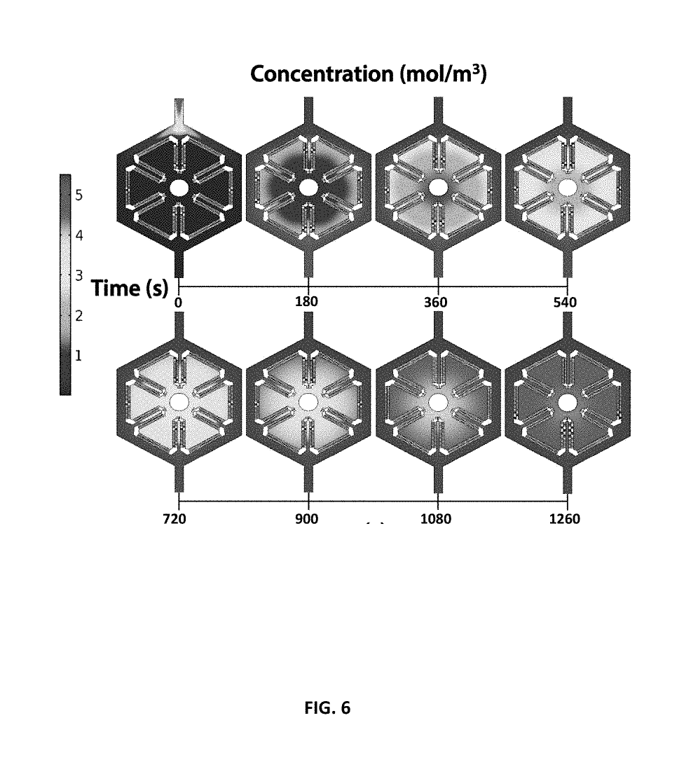

The transport of glucose molecules into the tissue culture chambers 30 via the feed channel 50 and the diffusion channels 42 was also simulated. The simulation is depicted in FIG. 6. The simulation was performed on a single chamber 30 with the same dimensions as in a device with a plurality of chambers 30. The simulations show that glucose diffusion under a 1 μl/min flow rate reaches the centre of the lobules in 120 seconds. The diffusion continues until the concentration reaches a steady state as shown in FIG. 6. Based on these simulations and the total volume of each chamber 30 (˜0.2 μl) and the total volume of the whole bottom feed network (˜7.3 μl) the flow rate was set to 1 μl/min in a 15-minute interval setting. This flow setting allows for the total volume of the feed network to be completely exchanged twice during the flow time and for the produced metabolites and cell waste to diffuse out of the chambers 30.

An advantage of the microfluidic devices described herein is that the devices provide a very large surface area of human liver tissue (more so than existing devices) that can provide statistical data and more accurate results on minute amounts of specific metabolites. The microfluidic devices can expand or shrink in a radial manner to represent the physiologically relevant size of the liver for different age and gender groups in a multi-organ platform. The microfluidic devices are capable of co-culturing hepatocytes with other cell types for example, liver endothelial cells present in the liver structure.

The microfluidic devices provide the maximum cell-to-cell interaction for the liver hepatocytes thereby significantly improving the liver-specific functionalities and mimicking the physiologically relevant niche of the liver. The controlled flow condition provides a constant supply of nutrients while washing away the secreted cell waste.

Endothelial-like PDMS walls 40 ensure that the 3D liver tissue in the chambers 30 receives fresh media at all times via designated diffusion channels 42. This ensures that liver cells will not be exposed to the shear stress induced by the flow of nutrients and mimics the physiological based blood stream inside the liver tissue. In the liver structure, liver-specific fenestrated endothelial cells reside between the blood stream and the hepatocytes and allow for the diffusion of nutrients and oxygen to the hepatic niche while protecting the hepatocytes from direct shear rate of the blood stream.

The microfluidic devices described herein may be used for acute and long-term drug toxicity and efficacy experiments. The metabolism of pharmaceutical or xenobiotics by cells cultured in the device 10, 20 is also provided. Cells cultured in the device may be provided with a xenobiotic, pharmaceutical etc. Metabolites from the cells may be extracted thereafter. The metabolites may be extracted from the central aperture 32 of each chamber 32. The metabolites may be extracted from the feed channel network 50, such as the outlets 80. The metabolites may be extracted from both he feed channel network 50 and the central aperture of each chamber 30.

Although, the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A microfluidic device for culturing and/or analysing at least one cell type comprising:
a plurality of chambers for a first cell type, arranged in a radial expansion model, each chamber having a central aperture for receiving the first cell type into the chamber and/or removing the first cell type from the chamber;
a wall on the perimeter of each chamber ;
a feed channel outside each chamber adjacent to the wall, forming a feed channel network for conveying culture medium, reagents, and/or a second cell type;
the feed channel network being provided with a central port and configured such that culture medium, reagents, and/or a second cell type provided to the central port are distributed symmetrically in a radial fashion in the feed channel network; and,
wherein the wall has a plurality of diffusion channels for allowing flow of the culture medium, reagents, and/or the second cell type from the feed channel into each chamber.

2. The microfluidic device according to claim 1, wherein the feed channel network extends radially outward from the central port towards the periphery of the plurality of chambers.

3. The microfluidic device according to claim 1, wherein the feed channel network is continuous.

4. The microfluidic device according to claim 1, wherein the plurality of chambers is arranged in adjacent concentric rings and wherein the feed channel network extends between the concentric rings.

5. The microfluidic device according to claim 1, wherein the plurality of chambers, wall, and feed channel network are located on a first layer; and
the device further comprises:
a second layer located adjacent to, and in reversible connection with, the first layer, the second layer comprising:
an inlet coinciding with the central port of the feed channel network for providing the culture medium and/or reagents to the feed channel.

6. The microfluidic device according to claim 5, wherein the second layer further comprises:
a plurality of openings coinciding with each central aperture for feeding the first cell type into the chamber and/or receiving the first cell type from the chamber.

7. The microfluidic device according to claim 5, wherein the inlet coinciding with the feed channel network is located centrally on the second layer.

8. The microfluidic device according to claim 5, further comprising channels extending from at least two of the openings to at least one main opening on the second layer.

9. The microfluidic device according to any of claim 1, further comprising at least one outlet on the periphery of the device for receiving the culture medium and/or reagents from the feed channel network.

10. The microfluidic device according to claim 1, wherein the diffusion channels are microfluidic diffusion channels having a width of from 2 μm to 10 μm, preferably about 2 μm.

11. The microfluidic device according to claim 1, wherein the diameter of each chamber is similar to the diameter of a mammalian liver lobule, such as a human liver lobule, preferably the diameter of each chamber is about 1 mm to about 2.5 mm, or more preferably about 1.2 mm to about 2.4 mm.

12. The microfluidic device according to claim 1, wherein each chamber comprises a plurality of cell culture compartments extending radially from the central aperture towards the wall forming a plurality of lobules, in a flower-petal like arrangement.

13. The microfluidic device according to claim 1, further comprising a plurality of posts in each chamber.

14. The microfluidic device according to claim 1, wherein the wall and the posts comprise a biocompatible polymer.

15. The microfluidic device according to claim 1, wherein at least 3 layers of cells can be cultivated within each chamber, forming a 3D tissue-like structure.

16. The microfluidic device according to claim 1 for co-culturing at least two different cell types, wherein the first cell type is cultured in a low shear flow environment, preferably the chamber, and the second cell type is cultured in a higher shear flow environment, relative to the low shear flow environment, preferably the feed channel.

17. The microfluidic device according to claim 1, wherein the first cell type is a hepatocyte, brain cell (neuron, glial cell), cardiac muscle cell (cardiomyocyte), lung epithelial cell (alveolar), intestinal epithelial cell, ovarian cell, fat cell (adipocytes), renal proximal tubule epithelial cell or bone marrow cell.

18. The microfluidic device according to claim 1, wherein the second cell type is a liver endothelial cell, capillary blood vessel cell, brain endothelial cell, lung endothelial cell, fibroblast cell, retinal vascular endothelial cell, kidney (renal) cell, Kupffer cell, hepatic stellate cell or microvascular endothelial cell.

19. A system for culturing cells comprising at least two microfluidic devices according to claim 5 wherein each of the microfluidic devices comprises a central inlet/outlet arranged in cooperation with a channel connecting at least two of the devices.

20. A method of culturing cells in a microfluidic device according to claim 1, comprising:
providing a first cell type to the central aperture of each of the plurality of chambers,
providing a culture medium to the central aperture and/or to the central port of the microfluidic device such that the first cell type is exposed to the culture medium.

21. The method of culturing cells according to claim 20, further comprising:
providing a second cell type to the central port of the microfluidic device such that the second cell type is distributed in a radial fashion throughout the feed network.

22. A method of analysing pharmaceutical/xenobiotic toxicity and/or efficacy on a culture of cells in a microfluidic device according to claim 1, the method comprising:
culturing cells in the microfluidic device, further comprising:
providing a first cell type to the central aperture of each of the plurality of chambers,
providing a culture medium to the central aperture and/or to the central port of the microfluidic device such that the first cell type is exposed to the culture medium;
providing said drug to the central port of the feed network; and,
extracting cell metabolites from each respective central aperture of the plurality of chambers and, optionally, the feed network.

23. The method of analysing pharmaceutical/xenobiotic toxicity and/or efficacy on a culture of cells in a microfluidic device according to claim 22, wherein culturing cells in the microfluidic device, further comprises:
providing a second cell type to the central port of the microfluidic device such that the second cell type is distributed in a radial fashion throughout the feed network.
